(19) United States
(12) Patent Application Publication
(10) Pub. No.: US 2025/0213497 A1
(43) Pub. Date: Jul. 3, 2025

(54) A NANO IN MICRO FORMULATION AND PROCESS THEREOF

(71) Applicant: AMRITA VISHWA VIDYAPEETHAM, Kochi, Kerala (IN)

(72) Inventors: MANZOOR KOYAKUTTY, Kochi, Kerala (IN); LEKSHMI GOPAKUMAR, Kochi, Kerala (IN); SHALIN CHAPPAN, Kochi, Kerala (IN); MAYA SREERANGANATHAN, Kochi, Kerala (IN); SHANTIKUMAR NAIR, Kochi, Kerala (IN)

(21) Appl. No.: 18/852,982

(22) PCT Filed: Mar. 29, 2023
(86) PCT No.: PCT/IN2023/050305

§ 371 Date: Sep. 30, 2024

(30) Foreign Application Priority Data
Mar. 30, 2022 (IN) 202241019008

(51) Int. Cl. A61K 9/51; A61K 31/44; A61K 31/495

(52) U.S. Cl. CPC A61K 9/5169; A61K 9/5153; A61K 9/5161; A61K 31/44; A61K 31/495

(57) ABSTRACT

The present invention relates to a nano-in micro formulation, capable of enhancing the oral bioavailability of drugs. Said formulation is made of a drug loaded composite nanoparticles made of protein-polymer, which is embedded within a microsystem (nano-in micro) made of multiple layers, each layer serving specific function such as enteric protection, muco-adhesion, muco-penetration, release modification, efflux inhibition, all functions collectively resulting in the enhanced bioavailability of loaded drug by at least two-fold when compared with control free drug. The present invention also discloses the process for conceiving said nano-in micro formulation and describes the use of said nano-in micro formulation in the treatment of cancer and other diseases.

### FIELD OF THE INVENTION

The present invention is in the field of drug formulations. It relates to multi-layered nano-in micro particle formulation also referred as formulation, suitable for oral delivery of drugs, having enhanced oral bioavailability, its process of production and its uses.

### BACKGROUND OF INVENTION

Oral-delivery of drug is the most preferred route of administration due to its non-invasive nature and better acceptability across age-groups. However, oral bioavailability is an important parameter considered in a pharmaceutical lead molecule development. Oral bioavailability is related to the drug concentration in the blood plasma after oral intake, and this in turn, is related to the release of drug from the dosage form, solubility of the drug, absorption in gastrointestinal tract, drug metabolism and clearance rate from the body. Oral absorption can be defined as the percentage of drug that survives gastrointestinal metabolism, passes through the intestinal tissue and enters the systemic circulation. Once entered into the blood stream, the drug can be metabolised and further cleared by hepatic or renal route. Low absorption and solubility of the drug along with fast clearance will result into inferior bioavailability. Drugs with poor solubility and low intestinal permeability, falling under the biopharmaceutical classification system (BCS) class II and IV show low oral bioavailability. Some of the BCS class III category drugs also show low bioavailability due to the low permeability across the intestinal tissue. Intestinal permeability is affected mainly because of the role of transporter proteins (ABC family proteins) such as P-Gp (P-glycoprotein). These drug efflux pumps impose a major barrier in the intestinal absorption of drugs, thereby affecting the bioavailability. All these factors often impose the requirement of high-dose administration in order to achieve the therapeutic dose but lead to dose limiting toxicities (DLTs) which hinders the therapeutic efficacy. There is a need to develop a formulation particularly of drugs which has poor solubility and low intestinal permeability, to enhance their oral bioavailability. It could increase the therapeutic efficacy of formulated drugs at low drug dosage and thereby preventing drug limiting toxicities.

### OBJECTIVES OF THE INVENTION

The objective of the present invention is to provide a nano-in micro formulation particularly of drugs which has poor solubility and low intestinal permeability which could enhance the oral bioavailability of formulated drugs, by at least two-fold when compared to the bioavailability of free un-formulated drugs. The object is for providing a nano-in micro formulation to increase the therapeutic efficacy of formulated drugs at low drug dosage and thus prevent the drug related toxicities associated with over dosage.

### SUMMARY OF THE INVENTION

The present invention discloses a specific multi-component protein-polymer-carbohydrate, nano-in micro formulation comprising core nanoparticles and shell, wherein said core nanoparticles are coated with one or more layers of said shell, suitable for the oral delivery of small molecule drugs, having 2-3-fold enhanced oral bioavailability.

In one of the embodiments of said nano-in micro formulation, the core nanoparticles comprise drug dissolved in solvent I, carrier protein dissolved in solvent II, polymer; wherein the one or more layers of shell are made of substances comprising muco-adhesives, release modifiers and optionally, bioavailability enhancers and cross-linking agents In one of the specific embodiment of said nano-in micro formulation/formulation, protein carrier employed is phycocyanin, which along with polymer blends, form core nanoparticles, which are loaded with small molecule drugs, which are either coated with or embedded within layers of shell made of substances comprising muco-adhesives, release modifiers and optionally, bioavailability enhancers and cross-linking agents. In one of the embodiments of said nano-in micro formulation, sorafenib tosylate has been employed as an embodiment of drug of interest, in order to demonstrate the bioavailability enhancement of the disclosed nano-in micro formulation. Sorafenib tosylate, which is a multi-kinase inhibitor, used for the treatment of advanced stage hepatocellular carcinoma, renal cell carcinoma and differentiated thyroid cancers, was specifically chosen as a drug of choice because of its high lipophilicity and its relative oral bioavailability is ˜38% and it also showed high interpatient variability in terms of oral bioavailability which imposed high daily dose of 800 mg to achieve the intended therapeutic efficacy. The high daily dose requirement is associated with dose limited toxicities which causes inferior patient compliance. The multi-component protein-polymer-carbohydrate, nano-in micro formulation of the present invention, formulated for oral delivery of sorafenib tosylate improved its bioavailability by at least two-fold. The present invention also discloses method of production of multi-component protein-polymer-carbohydrate, nano-in micro formulation comprising inline mixing, colloidal wet milling or homogenisation at ambient pressure and room temperature. The present invention also encompasses the use of said multi-component protein-polymer-carbohydrate, nano-in micro formulation in anticancer therapy, anti-inflammatory therapy, antipyretic, anti-hypertensive, antihistamines, anti-epileptic, anti-diabetic, upper respiratory tract infections, hormonal replacement therapy, bacterial and fungal infections, arthritis, cardiovascular diseases, neuronal/degenerative disorders, auto immune diseases.

### ADVANTAGES OF THE PRESENT INVENTION

The multi-component protein-polymer-carbohydrate, nano-in micro formulation of present invention enhances the bioavailability or the plasma concentration of the formulated drug, sorafenib for instance by at least 2-fold when compared to control unformulated drug. Further, it was also observed that the nano-in micro formulation of present invention also exhibited improved antitumor efficacy than the control unformulated drug resulting in complete elimination of the subcutaneous tumors at half the dosage as that of control unformulated drug.

### BRIEF DESCRIPTION OF THE DRAWINGS

The invention has other advantages and features which will be more readily apparent from the following detailed description of the invention, when taken in conjunction with the accompanying drawings, in which:

FIG. 1: Diagrammatic representation of method of preparation of final formulation according to some embodiments using stirrer.

FIG. 2: Schematic representation of the method of preparation of final formulation according to some embodiments using stirrer.

FIG. 3: Diagrammatic representation of method of preparation of final formulation according to some embodiments using high pressure homogeniser.

FIG. 4: Schematic representation of the method of preparation of final formulation according to some embodiments using high pressure homogeniser.

FIG. 5: Diagrammatic representation of method of preparation of final formulation according to some embodiments using wet milling unit.

FIG. 6: Schematic representation of the method of preparation of final formulation according to some embodiments using wet milling unit.

FIG. 7: Pharmacokinetic profile of 41.1 mg/kg of phycocyanin sorafenib nano formulation-1 compared to that of clinical control sorafenib following an oral administration in healthy SD rats.

FIG. 8A: Size distribution of phycocyanin sorafenib nanoparticle after 10 cycles of high-pressure homogenization using Dynamic Light Scattering FIG. 8B: Transmission electron microscopic image (TEM) of phycocyanin-sorafenib nanoparticle.

FIG. 8C: Scanning electron microscopic image (SEM) of phycocyanin-sorafenib nanoparticle.

FIG. 8D: Size distribution of phycocyanin-sorafenib nano formulation-2 after coating with blend of bioavailability enhancer, muco-penetrating and release modifying polymeric layer.

FIG. 8E: TEM image of phycocyanin-sorafenib nano formulation-2 after coating.

FIG. 8F: SEM of phycocyanin-sorafenib nano formulation-2 after crosslinking into micro matrix structure (nanoparticles shown with arrows).

FIG. 9A: Pharmacokinetics of 41.1 mg/kg of phycocyanin-sorafenib nano formulation-2 compared with control sorafenib following single oral administration in healthy SD rats.

FIG. 9B: Plasma concentration profile for sorafenib after continuous multiple doses of phycocyanin-sorafenib nano formulation-2 and control sorafenib for 12 days.

FIG. 10: Tumor reduction profile of subcutaneous AML (MV-4-11) xenografts following alternate day treatment with 41.1 mg/kg of control sorafenib and phycocyanin-sorafenib nano formulation-2 in nude mice models.

FIG. 11: Pharmacokinetic profile of 20.55 mg/kg of phycocyanin-sorafenib nano formulation-3 compared to that of 41.1 mg/kg of control following an oral administration in healthy SD rats.

FIG. 12: Pharmacokinetic profile of 20.55 mg/kg of albumin-sorafenib nano formulation-1 compared to that of 41.1 mg/kg of control following an oral administration in healthy SD rats.

FIG. 13: Pharmacokinetic profile of 41.1 mg/kg of albumin-sorafenib nano formulation-2 prepared using stirrer compared to that of control following an oral administration in healthy SD rats.

FIG. 14A: The size distribution of albumin-sorafenib nanoparticle after 20 cycles of high-pressure homogenization using Dynamic Light Scattering.

FIG. 14B: Scanning electron microscopic image of albumin-sorafenib nanoparticles.

PCT-2408 6

FIG. 14C: The size distribution of albumin-sorafenib nano formulation-2 after coating with blend of bioavailability enhancer, muco-penetrating and release modifying polymeric layer.

FIG. 14D: TEM image of albumin-sorafenib nano formulation-2 after coating.

FIG. 14E: SEM image of albumin-sorafenib nano formulation-2 after crosslinking into micro matrix structure (nanoparticles shown with arrows)

FIG. 15: Table showing the size, zeta, encapsulation efficiency and loading efficiency of albumin-sorafenib nano formulation-2 (ABSORF NANOFORMULATIONS).

FIG. 16: Detachment force showing the mucoadhesive property of various polymer coated albumin-sorafenib nano formulations.

FIG. 17: Single dose pharmacokinetics of Albumin-sorafenib nanoparticle-2 (41.1 mg/kg) with and without mucoadhesive polymeric layers compared with control sorafenib (41.1 mg/kg) following an oral administration in healthy SD rats.

FIG. 18A: pharmacokinetics of 41.1 mg/kg of albumin sorafenib nano formulation-2 compared with control sorafenib following single oral administration in healthy SD rats.

FIG. 18B: Plasma concentration profile for sorafenib after continuous multiple doses of albumin sorafenib nano formulation-2 and control sorafenib for 12 days.

FIG. 19A: Tumor regression profile of orthotopic N1S1 liver tumors when treated with control sorafenib and albumin-sorafenib nano formulation-2.

FIG. 19B: MRI images of tumor animals post 16 days treatment.

FIG. 20: Pharmacokinetic profile of 20.55 mg/kg of albumin-sorafenib nano formulation-3 compared to that of 41.1 mg/kg of control following an oral administration in healthy SD rats.

FIG. 21: Pharmacokinetic profile of 20.55 mg/kg of albumin sorafenib nano formulation-4 compared to that of 41.1 mg/kg of control following an oral administration in healthy SD rats.

FIG. 22: Pharmacokinetic profile of albumin-temozolomide nano formulation compared to that of control temozolomide at a dose of 40 mg/kg following an oral administration in healthy SD rats.

FIG. 23A: The size distribution of casein-lenvatinib nanoparticle using Dynamic Light Scattering.

FIG. 23B: Scanning electron microscopic image of casein-lenvatinib nanoparticles.

FIG. 24A: The size distribution of zein-regorafenib nanoparticle using Dynamic Light Scattering

FIG. 24B: Scanning electron microscopic image of zein-regorafenib nanoparticles

FIG. 25: The size distribution of albumin-cabozantinib nanoparticle using Dynamic Light Scattering (insert showing the formed nanoparticles)

FIG. 26: The size distribution of albumin-lopinavir nanoparticle using Dynamic Light Scattering

FIG. 27: The size distribution of albumin-amphotericin nanoparticle using Dynamic Light Scattering

### DETAILED DESCRIPTION OF INVENTION

For the purposes of the following detailed description, it is to be understood that the invention may assume various alternative variations and step sequences, except where expressly specified to the contrary. Moreover, other than in any operating examples, or where otherwise indicated, all numbers expressing, for example, quantities of ingredients used in the specification are to be understood as being modified in all instances by the term “about”.

Before describing the present invention in detail, it is to be understood that this invention is not limited to particularly exemplified systems or process parameters that may of course, vary. It is also to be understood that the terminology used herein is for the purpose of describing particular embodiments of the invention only, and is not intended to limit the scope of the invention in any manner. The use of examples anywhere in this specification including examples of any terms discussed herein is illustrative only, and in no way limits the scope and meaning of the invention or of any exemplified term. Likewise, the invention is not limited to various embodiments given in this specification.

Unless otherwise defined, all technical and scientific terms used herein have the same meaning as commonly understood by one of ordinary skill in the art to which this invention pertains. In the case of conflict, the present document, including definitions will control.

It must be noted that, as used in this specification and the appended claims, the singular forms “a,” “an” and “the” include plural referents unless the content clearly dictates otherwise. Thus, for example, reference to a “polymer” may include two or more such polymers.

The terms “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful. Further it is not intended to exclude other embodiments from the scope of the invention. As used herein, the terms “comprising” “including,” “having,” “containing,” “involving” and the like are to be understood to be open-ended, i.e., to mean including but not limited to.

The terms “formulation” and “nano-in micro formulation” refers to the same substance and are interchangeably used throughout the specification.

The present invention discloses a specific formulation (100, 300, 500), also referred as nano-in micro formulation comprising: core nanoparticles (105, 305, 505) and shell; wherein said core nanoparticles (105, 305, 505) are coated with one or more layers of said shell; wherein the core nanoparticles (105, 305, 505) comprise drug (104, 304, 504) dissolved in solvent I, carrier protein (101, 301, 501) dissolved in solvent II, polymer (102, 103; 302, 303; 502, 503); wherein the one or more layers of shell are made of substances comprising muco-adhesives (107, 307, 507), release modifiers (108, 308, 508).

In particular embodiment of the formulation (100, 300, 500), said substances further comprise bioavailability enhancers (106, 306, 506) and cross-linking agents (110, 310, 510).

The formulation (100, 300, 500) is specially designed to enhance the intestinal absorption and solubility, while suppressing fast clearance, thus increasing the overall bioavailability of drugs. Specific nanocarriers including biodegradable polymer or proteins have been employed which shield the drugs from fast degradation in acidic gastro-intestinal environment which also help in extended release of the drug into the plasma. To improve the intestinal permeability, permeation enhancers and drug efflux pump inhibitors have been employed to increase the bioavailability.

The nano-in micro formulation (100, 300, 500) of the present invention for enhancing the oral bioavailability of drug uses a unique nano-microstructure comprising core nanoparticles which in turn comprise a carrier protein, drug, polymer and whose surface is coated with layers made of substances such as muco-adhesives and muco-penetrants or release modifiers. Said nano-in micro formulation (100, 300, 500) is a specific multi-component formulation comprising composite core nanoparticles made of protein-polymer-carbohydrate, wherein the drug is loaded into the protein part and the polymer part provides drug solubilization and muco-penetration properties. Further these composite nanoparticles are embedded within a polymeric microsystem having muco-adhesion and enteric coating properties. This drug loaded nano-in micro formulation (100, 300, 500) is suitable for oral delivery of small molecule drug, including sorafenib, with 2 to 3-fold enhanced oral bioavailability which thereby improves the therapeutic efficacy of the loaded drug.

Various embodiments of the nano-in micro formulation (100, 300, 500), further comprises binders, fillers, disintegrating agent, glidants, lubricants, sorbents, preservative, excipients, stabilizers, solubilizers and flavouring agents suitable for making capsule or tablet or suspension suitable for oral delivery.

In an embodiment of the formulation (100, 300, 500), the ratio of drug (104, 304, 504) dissolved in solvent I and carrier protein (101, 301, 501) dissolved in solvent Il in the core nanoparticles is 1:1.

In various embodiments of the formulation (100, 300, 500), the core nanoparticles (105, 305, 505) range in size between 1-1000 nm, preferably between 1-200 nm, 200-500 nm or 500-1000 nm and wherein particles of the final formulation (100, 300, 500) range in size between 1-1000 μm, preferably between 1-5 μm, 5-100 μm, 100-1000 μm.

In various embodiments of the formulation (100, 300, 500), the polymer (102, 103, 302, 303, 502, 503) is in the range of 10% to 50% by weight of carrier protein (101, 301, 501) in the core nanoparticles (105, 305, 505); wherein the one or more layers of shell comprise muco-adhesives (107, 307, 507) in the range of 20-40% by weight of drug (104, 304, 504); release modifiers (108, 308, 508) in the range of 1-15% by weight of drug (104, 304, 504); bioavailability enhancers (106, 306, 506) in the range of 8.5-10% by weight of drug (104, 304, 504); cross-linking agents (110, 310, 510) in the range of 5%-20% by weight of drug (104, 304, 504).

In various embodiments of the formulation (100, 300, 500), the drug (104, 304, 504) is present in the range of 5 mg/ml-100 mg/ml of the final formulation (100, 300, 500).

In one of the embodiments of said nano-in micro formulation (100, 300, 500), Phycocyanin is employed as the carrier protein, wherein the core nanoparticle is loaded with small molecule drugs and, preferably added with bioavailability enhancing molecules, and the core is coated or embedded within muco-adhesive and muco-penetrating polymer and/or carbohydrate layers. Such a unique composite formulation is capable of providing 2 to 3-fold enhanced plasma concentration of the drug post oral delivery. Phycocyanin, which is a photosynthetic protein is extracted from light-harvesting chromoproteins.

In various other embodiments of the nano-in micro formulations (100, 300, 500), the carrier protein (101, 301, 501) that could be employed, is selected from phycocyanin, bovine serum albumin, human serum albumin, fibrinogen, collagen, gelatin, casein, mucin, protamine, transferrin, soy protein, apoferritin, ferritin, lectin, lactoferrin, gluten, whey protein, prolamines such as gliadin, hordein, secalin, zein, avenin or their salts.

In yet other embodiments of the nano-in micro formulation (100, 300, 500), the carrier protein (101, 301, 501) that could be employed could also be used in combination with lipids or fatty acids such as lecithin, cholesterol, coconut oil, corn oil, soybean oil, canola or rapeseed oil, sunflower oil, sesame oil, linseed oil, arachis oil, cottonseed oil, coconut or palm oil (mono-, di-, triglycerides), mixture of mono-and diglycerides of caprylic/capric acid, propylene glycol monocaprylate, glycerol caprylate caprate, glyceryl mono-dicaprylate1, 2, 3-propanetrioldecanoic acid monoester, oleic acid/ethyl oleate.

In various embodiments of the nano-in micro formulation (100, 300, 500), the drug (104, 304, 504) that is loaded into core nanoparticles includes but not limited to sorafenib, everolimus, rapamycin, tandutinib, sunitinib, lestaurtinib, semaxinib, midostaurin, nilotinib, dasatinib, imatinib, temsirolimus, lapatinib, vorinostat, bosutinib, olaparib, erlotinib, epirubicin, gefitinib, daunorubicin, temozolomide, nintedanib, crizotinib, dabrafenib, vemurafenib, ibrutinib, axitinib, regorafenib, ponatinib, cabozantinib, alectinib, brigatinib, lorlatinib, encorafenib, acalabrutinib, vandetanib, cobimetinib, lenvatinib, binimetinib, ceritinib, pazopanib, tacrolimus, amphotericin, bendamustine, methotrexate, cyclosporin, febuxostat, bromohexin, salbutamol, azathioprine, doxylamine, fexofenadine, bilastin, levocetrizine, cinnarizine, promethazine, terbutaline, doxofylline, loratadine, ibuprofen, naproxen, ketoprofen, piroxicam, aceclofenac, meloxicam, Etodolac, eterocoxib, celecoxib, indomethacin, carbamazepine, diazepam, clonazepam, lamotrigine, oxcarbazepine, gabapentin, perampanel, phenytoin, acetaminophen, ketoprofen, nimesulide, metformin, acetohexamide, chlorpropamide, tolazamide, tolbutamide, glibenclamide/gliburide, glipizide, glimepiride, gliclazide, repaglinide, nateglinide, dapagliflozin, rosiglitazone, pioglitazine, acarbose, exenatide, liraglutide, saxagliptin, bactrim, sulfamethoxazole, ceftin, biaxin, trimethoprim and sulfamethoxazole, ampicillin, cefpodoxime, penicillin, tetracycline, erythrocin, amoclan, bicillin c, dynapen, erythrocin stearate, vancomycin, trimethoprim, estrogen, progesterone, testosterones, thyroxine, insulin, prolactin, serotonin, cortisol, adrenaline, growth hormones, dabigatran, apixaban, rivaroxaban, vilazodone, ace inhibitors, angiotensin ii receptor blockers, beta-blockers, statins, anti-anginal medicines, amantadine, apomorphine, baclofen, carbidopa, dantrolene, entacapone, rasagiline, riluzole, rivastigmine, ropinirole, selegiline, tetrabenazine, tizanidine, tolcapone, terbinafine, Ciprofloxacin, Norfloxacin, azithromycin, azithromycin dihydrate, cefuroxime, cefixime, cefpodoxime proxetil, cefdinir, chloramphenicol, erythromycin, clarithromycin, Isoniazid, rifampin, pyrazinamide, ethambutol, prothionamide, linezolid, dapson (diaminodiphenyl sulfone), Ketoconazole,

Fluconazole, itraconazole, Terbinafine, Acyclovir, tenofovir, valaciclovir, oseltamivir, Zidovudine, etravirin, raltegravir, Mefloquine, enalapril, irbesartan, valsartan, olmesartan, telmisartan, candesartan, eprosartan.

In various embodiments of the nano-in micro formulation (100, 300, 500), the drug (104, 304, 504) that is loaded into core nanoparticles preferably comprises small-molecule inhibitors and DNA alkylating agents; anti-fungal drugs, anti-viral drugs; wherein the small-molecule inhibitors comprise sorafenib, sorafenib tosylate, everolimus, rapamycin, tandutinib, sunitinib, lestaurtinib, semaxinib, midostaurin, nilotinib, dasatinib, imatinib, temsirolimus, lapatinib, vorinostat, bosutinib, olaparib, erlotinib, epirubicin, gefitinib, daunorubicin, temozolomide, nintedanib, crizotinib, dabrafenib, vemurafenib, ibrutinib, axitinib, regorafenib, ponatinib, cabozantinib, alectinib, brigatinib, lorlatinib, encorafenib, acalabrutinib, vandetanib, cobimetinib, lenvatinib, binimetinib, ceritinib, pazopanib, tacrolimus; wherein the DNA alkylating agents comprise temozolomide, wherein the anti-fungal drugs comprise amphotericin and anti-viral drugs comprise lopinavir.

Among various embodiments of the nano-in micro formulation (100, 300, 500), the polymer (102, 103; 302, 303; 502, 503) that could be present along with carrier protein in core nanoparticle includes but not limited to poly (D,L-lactide-co-glycolic), polycaprolactones, and poly(D,L-lactide). Said polymeric nanoparticles are employed for increasing oral bioavailability of drugs with poor solubility, chemical/enzymatic stability, and poor permeability. Polymer complexation with cyclodextrin, its derivatives, Polyethylene glycol (PEG), other graft polymers of polyethylene glycol such as PCL-PVAc-PEG graft copolymer could also be preferably employed. In various embodiments, polymer (102, 103; 302, 303; 502, 503) blended with carrier protein is selected from ethylene glycol, polypropylene glycol, polyethylene glycol (of molecular weights1-200 Da, 200-400 Da, 400-600 Da, 600-1000 Da, 1000-6000 Da, 6000-8000 Da, 8000-20000 Da, 20000-40000 Da), polyethylene glycol-poly ethylene oxide block copolymers, PCL-PVA-PEG block copolymer, poly (vinyl pyrrolidone), poly (acrylamide) and copolymers, poloxamer, polymers of hydroxy acids such as polylactide, polyglycolide and polycaprolactone; polyanhydrides; polyortho esters; polyalkenes, such as polyethylene and polypropylene; substituted polyalkenes, such as polystyrene; poly-caprolactone (PCL), poly-valerolacton (PVL), poly-hydroxy butyrate (PHB), poly vinyl alcohol (PVA) poly-hydroxyvalerate (PHV), polyvinylpyrrolidone (PVP), Polyethyleneimine (PEI), poly sialic acid and lactide/trimethylene carbonate copolymers or combination thereof.

In specific embodiments of the nano-in micro formulation (100, 300, 500), the polymer (102, 103; 302, 303; 502, 503) is selected from polyethylene glycol 400 (PEG-400), block co-polymer of polyvinyl caprolactum-polyvinyl alcohol-polyethylene glycol (PCL-PVA-PEG) or combination thereof.

In a preferred embodiment of the nano-in micro formulation (100, 300, 500), the polymer (102, 103; 302, 303; 502, 503) is selected from polyethylene glycol 400 (PEG-400), block co-polymer of polyvinyl caprolactum-polyvinyl alcohol-polyethylene glycol (PCL-PVA-PEG) wherein the ratio of PEG-400: PCL-PVA-PEG is 1:5. In various embodiments of the nano-in micro formulation (100, 300, 500), the solvent I is one selected from water or organic solvent comprising dimethyl sulfoxide (DMSO),Imethanol, ethanol, dimethyl formamide, acetonitrile, dioxane and the solvent II is one selected from an organic solvent comprising dimethyl sulfoxide (DMSO), methanol, ethanol, dimethyl formamide, acetonitrile, dioxane or combination of organic solvent and polymer comprising PEG-400.

In various embodiments of the nano-in micro formulation (100, 300, 500), the bioavailability enhancer (106, 306, 506) is selected from piperlongumine, piperine, boswellic acid extracts, 3-acetyl-11-keto-β-boswellic acid, soluplus etc.,

These are alkaloids, slightly soluble in water and highly soluble in alcohol, chloroform and ether.

In preferable embodiments of the nano-in micro formulation (100, 300, 500), the bioavailability enhancer (106, 306, 506) is selected from piperlongumine, piperine, 3-acetyl-11-keto-β-boswellic acid.

In various embodiments of the nano-in micro formulation (100, 300, 500), the muco-adhesives (107, 307, 507) include but not limited to galactomannan, which is a polysaccharide consisting of a mannose backbone with galactose side groups, gelatine, alginate and its salts preferably sodium alginate, polymethacrylate copolymers such as eudragit, chitosan, carboxymethyl chitosan, thiolated chitosan, hydrophobic cellulosic polymers, such as ethylcellulose; blends of these polymers; and copolymers formed of the monomers of these polymers.

In various embodiments of the nano-in micro formulation (100, 300, 500), the muco-adhesives (107, 307, 507) preferably comprises galactomannan, alginate, preferably sodium alginate.

In various embodiments of the nano-in micro formulation (100, 300, 500), the release modifier (108, 308, 508) includes but not limited to HPMC (Hydroxy propyl methyl cellulose), poly (adipic anhydride), polyfumaric anhydride, polysebacic anhydride, polymaleic anhydride, polymalic anhydride, polyphthalic anhydride, polyisophthalic anhydride, polyaspartic anhydride, polyterephthalic anhydride, polyisophthalic anhydride, polycarboxyphenoxypropane anhydride and copolymers with other polyanhydrides, Poly (acrylic acid) (PAA), methylcellulose (MC), ethylcellulose (EC), hydroxyethyl cellulose (HEC), hydroxypropyl cellulose (HPC), hydroxypropyl methylcellulose (HPMC), hydroxypropyl methacrylate, and carboxymethyl cellulose (CMC), glycerol, pectin, polyethylene glycol, sorbitol, maltitol, mannitol, hydrogenated glucose syrups, xylitol, polydextrose, glyceryl triacetate, propylene glycol, propylene glycol alginate, fenugreek gum, gaur gum, tara gum, locust bean gum, cassia gum or combination thereof.

In various embodiments of the nano-in micro formulation (100, 300, 500), the release modifier (108, 308, 508), preferably comprises methylcellulose (MC). ethylcellulose (EC), hydroxyethyl cellulose (HEC), hydroxypropyl cellulose (HPC), hydroxypropyl methylcellulose (HPMC), hydroxypropyl methacrylate, and carboxymethyl cellulose (CMC), hydroxypropyl methyl cellulose phthalate (HPMCP), polypropylene glycol, poly methyl acrylates selected from Eudragit RS100, RS PO, RS 30D, RL100, RL PO, RL 30D, NE30D, and NE40D.

In a specific embodiment of the nano-in micro formulation (100, 300, 500), the release modifier (108, 308, 508) is preferably HPMC.

In various embodiments of the nano-in micro formulation (100, 300, 500), the cross-linking agent (110, 310, 510) comprises calcium chloride, glutaraldehyde.

In a specific embodiment of the nano-in micro formulation, the core nano particle comprises phycocyanin or albumin dissolved in water, polyethylene glycol, PCL-PVA-PEG block copolymer, sorafenib tosylate dissolved in DMSO; wherein phycocyanin or albumin dissolved in water and sorafenib tosylate dissolved in DMSO are present in the ratio of 1:1; and wherein the one or more layers of shell is formed of galactomannan, HPMC, piperlongumine.

In a specific embodiment of the nano-in micro formulation, the core nano particle comprises phycocyanin or albumin dissolved in water, polyethylene glycol, PCL-PVA-PEG block copolymer, sorafenib tosylate dissolved in DMSO; wherein phycocyanin or albumin dissolved in water and sorafenib tosylate dissolved in DMSO are present in the ratio of 1:1; and wherein the one or more layers of shell is formed of sodium alginate, HPMC, piperine, calcium chloride.

In a specific embodiment of the nano-in micro formulation, the core nano particle comprises albumin dissolved in water, polyethylene glycol, PCL-PVA-PEG block copolymer, temozolomide dissolved in DMSO; wherein albumin dissolved in water and temozolomide dissolved in DMSO are present in the ratio of 1:1; and wherein the one or more layers of shell is formed of sodium alginate, HPMC, piperine, calcium chloride.

In a specific embodiment of the nano-in micro formulation, the core nano particle comprises of phycocyanin dissolved in water, polyethylene glycol, PCL-PVA-PEG block copolymer, sorafenib tosylate dissolved in DMSO; wherein phycocyanin dissolved in water and sorafenib tosylate dissolved in DMSO are present in the ratio of 1:1; and wherein the one or more layers of shell is formed of galactomannan, HPMC.

In a specific embodiment of the nano-in micro formulation, the core nano particle comprises of albumin dissolved in water, polyethylene glycol, PCL-PVA-PEG block copolymer, sorafenib tosylate dissolved in DMSO; and wherein albumin dissolved in water and sorafenib tosylate dissolved in DMSO are present in the ratio of 1:1; wherein the one or more layers of shell is formed of sodium alginate, HPMC, calcium chloride.

In a specific embodiment of the nano-in micro formulation, the core nano particle comprises of albumin dissolved in water, polyethylene glycol, PCL-PVA-PEG block copolymer, sorafenib tosylate dissolved in DMSO; and wherein albumin dissolved in water and sorafenib tosylate dissolved in DMSO are present in the ratio of 1:1; wherein the one or more layers of shell is formed of 3-acetyl-11-keto-β-boswellic acid, galactomannan, HPMC.

In various embodiments, the nano-in micro formulation is used as standalone drug or in combination with other anti-cancer drugs.

A specific embodiment of the nano-in micro formulation consisting sorafenib as drug, is used for the treatment of hepatocellular carcinoma, renal cell carcinoma, acute myeloid leukemia, thyroid cancer, non alcoholic steatohepatitis and hepatic fibrosis for which administered dosage is in the range of 50 mg, 100 mg, 200 mg, 400 mg, 600 mg, 800 mg.

Yet another specific embodiment of the nano-in micro formulation consisting of temozolomide as drug is used for the treatment of glioblastoma for which administered dosage is in the range of 5 mg, 20 mg, 50 mg, 100 mg, 140 mg, 180 mg, 250 mg.

In a specific embodiment, the nano-in micro formulation (100, 300, 500), is in the form of oral powder formulation.

Another aspect of the invention pertains to a method of preparation of formulation (100, 300, 500) said method comprising:
dissolving carrier protein (101, 301, 501) in solvent I and blending with polymer (102, 103; 302, 303; 502, 503) to prepare composite carrier material.
dissolving drug (104, 304, 504) in solvent Il to provide drug solution;
drop wise addition of the drug solution into the composite carrier material under constant stirring to form nanoparticle suspension;
dissolving muco-adhesive (107, 307, 507) and release modifier (108, 308, 508) in organic solvent to form solution;
homogenizing said solution with nano-particle suspension, to form a homogenate;
collecting the homogenate and optionally blending with bioavailability enhancers (106, 306, 506) and/or cross-linking agents (110, 310, 510) and freeze drying to obtain formulation (100, 300, 500); wherein solvent I comprises water or organic solvent comprising dimethyl sulfoxide (DMSO), methanol, ethanol, dimethyl formamide, acetonitrile, dioxane and solvent Il is an organic solvent comprising dimethyl sulfoxide (DMSO), methanol, ethanol, dimethyl formamide, acetonitrile, dioxane or a combination of organic solvent and polymer comprising PEG-400.

In various embodiments of the method, the homogenization step is performed by using a stirrer, or by using a high-pressure homogeniser (HPH), subjecting to homogenisation cycles ranging from 1-100 cycles, pressure ranging from 5000 PSI-40,000 PSI or by using a wet milling unit and subjecting to homogenisation cycles ranging from 10-100 cycles.

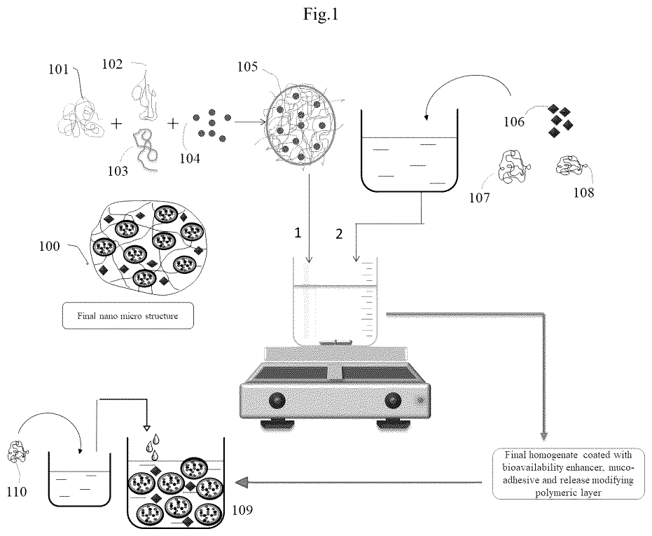

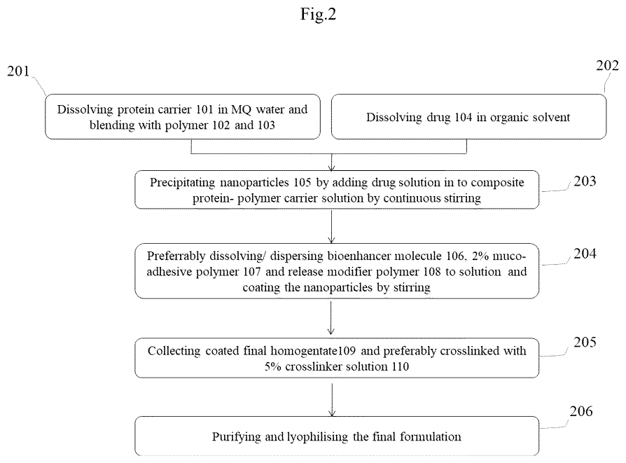

In a specific embodiment of the process for the preparation of nano-in micro formulation (100), the steps for preparing nano-in micro formulation for oral delivery is shown in FIG. 1 and the process flow-chart in FIG. 2. The process comprises of selecting a carrier protein 101 which is first blended with polymer 102 and 103. The small molecule drug 104 dissolved in organic solvent is reacted with 101-102-103 blend under mechanical stirring to form the drug loaded nanoparticle core 105, as shown in FIG. 2. Respective process steps are 201, 202 and 203 (FIG. 2). In the embodiment, the as prepared nanoparticle core 105 is preferably mixed with a solution containing a selected bioavailability enhancer 106, muco-adhesive polymer 107 and a release modifier polymer 108, dissolved or blended in solvent as in step 204 and allowed to coat the nanoparticle by stirring continuously. As in step 205, the coated final homogenate 109 is collected and cross-linked, preferably using 5% cross-linker 110 and then purified and lyophilised as shown in step 206 to get the final nano formulation 100.

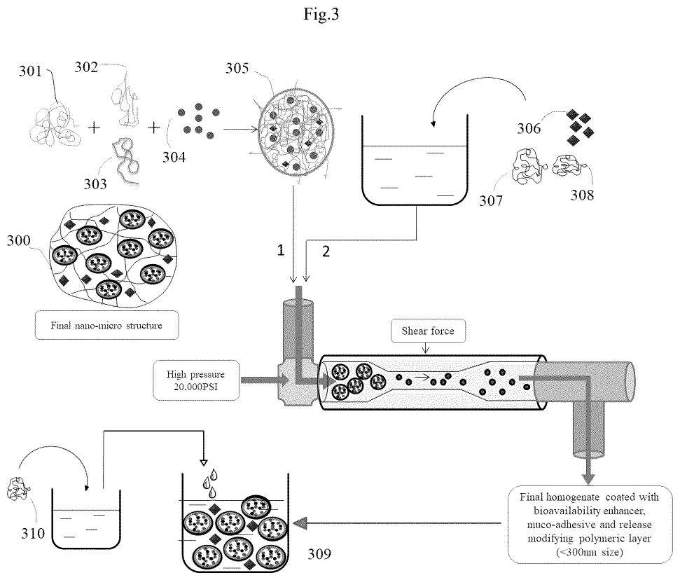

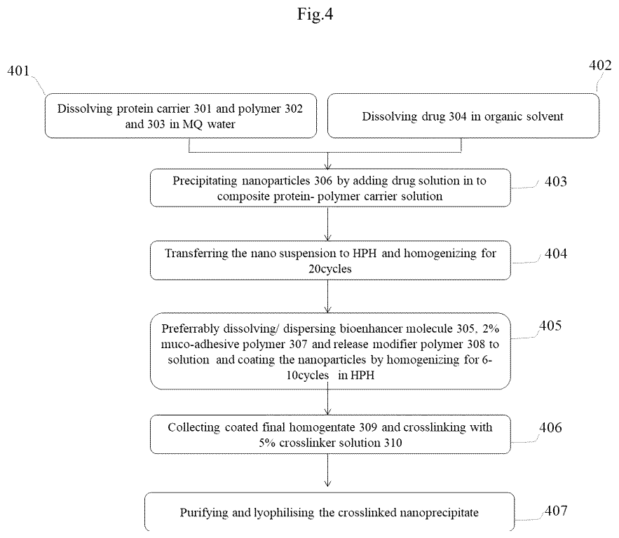

In yet another specific embodiment of the process for the preparation of nano-in micro formulation (300), the nano formulation 300 is prepared using a process involving high-pressure homogenization as shown in FIGS. 3 and 4. Said process involved the following steps: a carrier protein 301 is blended with polymers 302 and 303, and the small molecule drug 304 dissolved in organic solvent is reacted with 301-302-303 blend to form the nanoparticle core 305 under mechanical stirring as shown in FIG. 4, step 401, 402 and 403. In the embodiment, the as prepared nanoparticle 305 is preferably transferred to high pressure homogenizer for homogenization 404, under high pressure varying from 5000-40000 psi and 1-100 cycles. In step 405, the bioavailability enhancer 306, muco-adhesive polymer 307 and a release modifier polymer 308 are first mixed together in aqueous solvent and added to high-pressure-homogenizer during the homogenization process of nanoparticle core. As in step 406, the coated final homogenate 309 is collected and cross-linked, preferably using 5% cross-linker 310 and then purified and lyophilised as shown in step 407 to get the final nano formulation 300.

In various other embodiments of preparation of nano-in micro formulation, the drug loaded protein nanoparticle is formed by a method selected from precipitation, high-pressure homogenization, coacervation, self-assembly, cross-linking, spray drying, lyophilisation, electrospray, emulsion desolvation, snap injection etc. In some embodiments, the prepared nano formulation 100 is purified by filtration, centrifugation and lyophilisation.

In yet another specific embodiment of the preparation of nano-in micro formulation as shown in FIG. 1, a specific drug, sorafenib tosylate 104 is dissolved in DMSO (FIG. 4, step 402) and core nanoparticle 105 is prepared by drop wise addition (step 203) into composite carrier solution consisting of phycocyanin 101 s carrier protein and polyethylene glycol 102 and PCL-PVA-PEG (Polycaprolactum-Poly vinyl alcohol-Polyethylene Glycol) block copolymer 103 as polymer. In one another embodiment of said preparation process, albumin could be employed as the carrier protein. In the above embodiment, the prepared core nanoparticles 105 are further coated with a solution of bioavailability enhancer piperlongumine 106, galactomannan 107 as muco-adhesive and HPMC as release modifier by continuous stirring (step 204).

In various embodiments, the bioavailability enhancer 106 can preferably be 3-acetyl-11-keto-β-boswellic acid. In some of the embodiments there can be preferably no bioavailability enhancer. In various embodiments the other muco-adhesive polymer can be alginate.

In another embodiment, the process of core nanoparticle preparation and the process of polymer coating can be done at high pressure homogenisation as shown in flow-chart (FIG. 4); step 404 and 405. In various embodiments of the preparation process of nano-in micro formulation, sorafenib tosylate 304 is dissolved preferably along with bioavailability enhancer in organic solvents which is selected from ethanol, methanol, dimethyl sulphoxide, dimethyl formamide, acetonitrile, dioxane. In some embodiments, the concentration of sorafenib 304 ranges from 10 mg/ml to 100 mg/ml, concentration of carrier protein 301 ranges from 1 mg/ml to 10 mg/ml and concentration of polymer 302 and 303 ranges from 1-20%. In one of the embodiments, the homogenization is done for 20 cycles at 20,000 PSI. In various embodiments, the number of cycles may vary from 1 to 100and the pressure of the high-pressure homogenizer will range from 5000 PSI to 30,000 PSI. The coated final homogenate is collected and preferably cross-linked using 0.01% glutaraldehyde 310, purified, and lyophilised as shown in step 406 to get the final nano formulation 300.

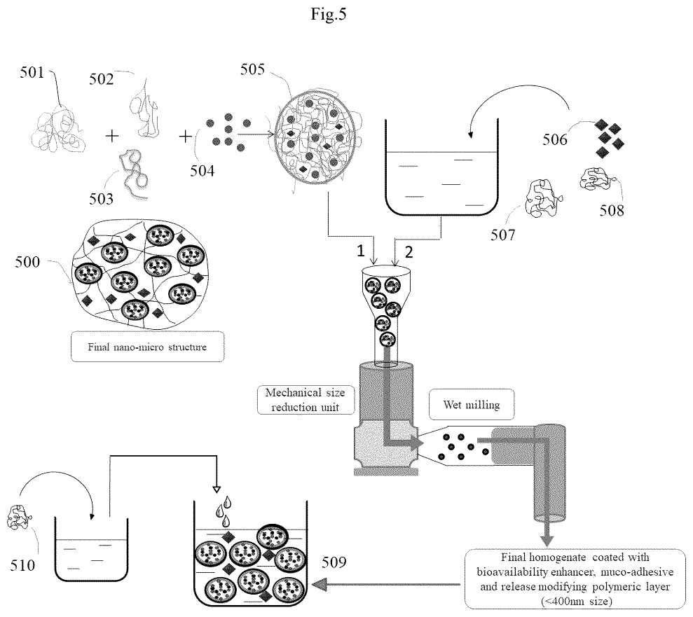

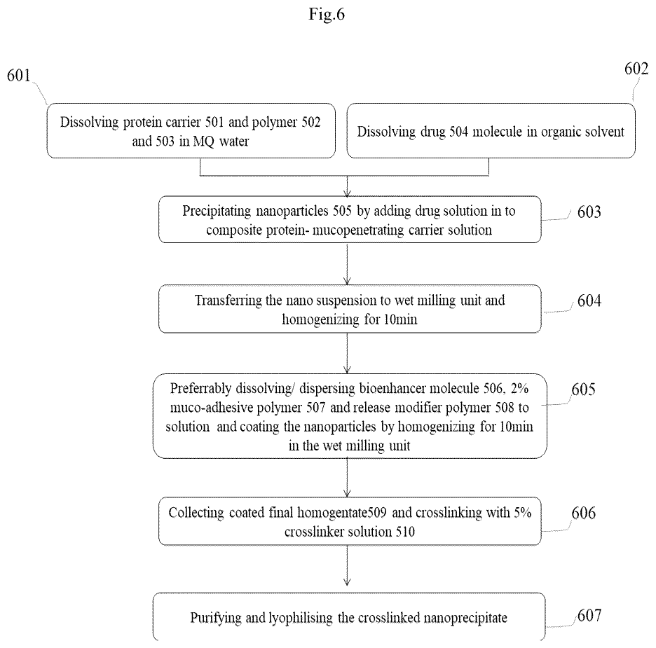

In yet another embodiment of the preparation process, wet-milling process is used as in FIGS. 5, and 6, wherein a carrier protein 501 is blended with a polymer 502 and 503, and the small molecule drug 504 dissolved in organic solvent is reacted with 501-502-503 blend to form the nanoparticle core 505 under mechanical stirring.

This is subjected to colloidal wet milling as in FIG. 6, step 604. In step 605, the bioavailability enhancer 506, muco-adhesive polymer galactomannan (alginate) 507 and release modifying polymer (HPMC) 508 is dissolved in solvent and added to the colloidal wet milling unit while the core nanoparticle is homogenized. As in step 606, FIG. 6, the coated final homogenate 509 is collected and preferably cross-linked using crosslinker 510 and then purified by filtration and lyophilised as shown in step 607 to get the final nano formulation 500.

In one of the embodiments, a specific carrier protein 101 is phycocyanin dissolved preferably in water at a concentration of 5 mg/ml. Polymers; PEG-400 (102) (10 wt % of the protein) and PCL-PVA-PEG block copolymer 103 (50% by weight of protein) is blended to the protein solution while stirring for total 10 minutes. Sorafenib tosylate 104 (1:1 ratio with respect to protein) is dissolved in DMSO. This mixture is added to phycocyanin solution drop-wise and stirred continuously for precipitation of nanoparticles 105 at 1200 rpm a room temperature. The prepared suspension can be preferably transferred to high pressure homogeniser (HPH) and homogenised under 20,000 psi for 20 cycles or transferred to colloidal wet milling for homogenisation. In the next step, the nanoparticles are coated by stirring with the solution of mucoadhesive layer 2 wt % solution of galactomannan 107, release modifier HPMC 108 and preferably along with bioavailability enhancer, piperlongumine 106. In some embodiment the coating can be done using high pressure homogenization 20,000 psi for 20 cycles (as in FIG. 4 step 405) or by using colloidal wet milling (as in fig.6; step 605) homogenization for 10 minutes. This mixture 109 or 309 is preferably cross-linked using crosslinker 110 solution by continuously stirring for 15mins, purified by filtration or centrifugation, and lyophilized to get the final formulation 100 or 300. The lyophilized powder is again preferably blended with piperlongumine a weight percentage of 8.5-10 wt % with respect to sorafenib to prepare the final formulation.

In yet another specific embodiment, albumin dissolved preferably in water at a concentration of 5 mg/ml is employed as specific carrier protein (101). Polymers; PEG-400 (102) (10 wt % of the protein) and PCL-PVA-PEG block copolymer 103 (50% by weight of protein) are blended to the protein solution while stirring for total 10 minutes. Sorafenib tosylate 104 (1:1 ratio with respect to protein) is dissolved in DMSO. This mixture is added to albumin solution dropwise and stirred continuously for precipitation of nanoparticles 105 at 1200 rpm a room temperature. The prepared suspension can be preferably transferred to high pressure homogeniser (HPH) and homogenised under 20,000 psi for 20 cycles or transferred to colloidal wet milling for homogenisation. In the next step, the nanoparticles are coated by stirring with the solution of mucoadhesive layer 2 wt % solution of galactomannan 107, release modifier HPMC 108 and preferably along with bioavailability enhancer, piperlongumine 106. In some embodiment the coating can be done using high pressure homogenization 20,000 psi for 20 cycles (as in FIG. 4 step 405) or by using colloidal wet milling (as in FIG. 6; step 605) homogenization for 10 minutes. This mixture 109 is preferably cross-linked using cross-linker 110 solution by continuously stirring for 15 mins, purified by filtration or centrifugation and lyophilized to get the final formulation 100 or 300. The lyophilized powder is again preferably blended with piperlongumine a weight percentage of 8.5-10 wt % with respect to sorafenib to prepare the final formulation.

Yet another aspect of the invention pertains to a method for increasing the bio-availability of a drug within a subject comprising administering therapeutically effective dose of the formulation (100, 300, 500).

In various embodiments of the method for increasing the bio-availability, the drug in the formulation comprises any one selected from sorafenib, sorafenib tosylate, everolimus, rapamycin, tandutinib, sunitinib, lestaurtinib, semaxinib, midostaurin, nilotinib, dasatinib, imatinib, temsirolimus, lapatinib, vorinostat, bosutinib, olaparib, erlotinib, epirubicin, gefitinib, daunorubicin, temozolomide, nintedanib, crizotinib, dabrafenib, vemurafenib, ibrutinib, axitinib, regorafenib, ponatinib, cabozantinib, alectinib, brigatinib, lorlatinib, encorafenib, acalabrutinib, vandetanib, cobimetinib, lenvatinib, binimetinib, ceritinib, pazopanib, tacrolimus, temozolomide, amphotericin and lopinavir.

In specific embodiments of the method for increasing the bio-availability, the drug is preferably sorafenib tosylate, temozolomide, lenvatinib, regorafenib, cabozatinib, amphotericin, lopinavir.

Yet another aspect of the invention pertains to a method of treating disease, comprising administering therapeutically effective dose of the formulation (100, 300, 500).

In various embodiments of the method of treating disease, the disease comprises cancer preferably of hepatocellular carcinoma, renal cell carcinoma, acute myeloid leukemia, thyroid cancer, glioblastoma, non-alcoholic steatohepatitis, hepatic fibrosis, fungal infections comprising candidiasis, viral infections caused by retro viruses.

In various embodiments of the method of treating disease, the therapeutically effective dose of formulation is in the range of 50 mg, 100 mg, 200 mg, 400 mg, 600 mg, 800 mg when the drug in said formulation is sorafenib tosylate; wherein the therapeutically effective dose of formulation is in the range of 5 mg, 20 mg, 50 mg, 100 mg, 140 mg, 180 mg, 250 mg when the drug in said formulation is temozolomide; wherein the therapeutically effective dose of formulation is in the range of 50 mg, 100 mg, 200 mg, 400 mg when the drug in said formulation is lopinavir.

The invention is further illustrated with reference to the following examples, which however, are not to be construed to limit the scope of the invention as defined by the appended claims.

### EXAMPLES

The materials used in the invention were purchased from various commercial sources as indicated below in Table 1 and 2. Swiss Albino nude mice and Sprague Dawley rats were obtained from Central Lab Animal Facility, Amrita Institute of Medical Sciences and Research Centre, Kochi (CPCSEA Reg .No. 527/02/A/CPCSEA-Dt21/01/2002. Renewal No: 527/PO/ReRcBi-S/ReRc-L/02/CPCSEA dated Jun. 5, 2022).

TABLE 1

| SI | | | |
|---|---|---|---|
| no | Protein | Biological Source | Company |
| 1 | Human serum | Human plasma | Sigma, USA |
| | albumin | | |
| 2 | Bovine serum | Bovine plasma | Sigma, USA |
| | albumin | | |
| 3 | Casein | Cow milk | Sigma, USA |
| 4 | Phycocyanin | Sea weeds | Synthite Industries Pvt, |
| | | (Aphanizomenon | Ltd, Kochi, India |
| | | sp., Spirulina sp., | |
| | | Phormidium sp., | |
| | | Lyngbya sp., | |
| | | Synechocystis sp. | |
| | | and | |
| | | Synechococcus | |
| | | sp) | |
| 5 | Soy protein | Soy beans | Sigma, USA |
| 6 | Whey protein | Milk | Sigma, USA |
| 7 | Gelatin | Skin, hides, bones | Sigma, USA |
| 8 | Fibrinogen | Human/bovine | Sigma, USA |
| | | plasma | |
| 9 | Mucin | — | Sigma, USA |
| 10 | Gluten | Wheat, barley | Sigma, USA |
| 11 | Gliadin | Wheat, barley | Sigma, USA |
| 12 | Zein | Corn gluten | Sigma, USA |
| 13 | Protamines | Fish sperm | Sigma, USA |
| 14 | Lectin | raw legumes | Sigma, USA |
| | | (beans, lentils, | |
| | | peas, soybeans, | |
| | | peanuts) and | |
| | | whole grains like | |
| | | wheat | |
| 15 | Lactoferrin | milk | Sigma, USA |
| 16 | Ferritin | legume seeds | Sigma, USA |
| | | (soybean, chick | |
| | | peas, lentils, | |
| | | lupine) | |
| 17 | Apoferritin | legume seeds | Sigma, USA |
| | | (soybean, chick | |
| | | peas, lentils, | |
| | | lupine) | |
| 18 | collagen | Animal origin | Sigma, USA |
| 19 | Transferrin | Animal origin | Sigma, USA |

TABLE 2

| SI no | Protein | Company |
|---|---|---|
| 1 | Piperine | Sigma, USA |
| 2 | Piperlongumine | Indofine, USA |
| 3 | 3-acetyl-11-keto-β-boswellic acid | Sigma, USA |
| 4 | Galactomannan | Sigma, USA |
| 5 | Soduim alginate | Sigma, USA |

### Example 1

### Preparation of Phycocyanin-Sorafenib Nano Formulation-1 Using Stirrer Method

Phycocyanin is dissolved preferably in water at a concentration of 5 mg/ml. Polymers; PEG-400 (10 wt % of the protein) and PCL-PVA-PEG block copolymer (50% by weight of protein) is blended to the protein solution while stirring for total 10 minutes. Sorafenib tosylate (1:1 ratio with respect to protein) is dissolved in DMSO. This mixture is added to composite carrier solution dropwise and stirred continuously for precipitation of nanoparticles at room temperature at 1200 rpm for 10 mimutes. In the next step, the nanoparticles are coated with a layer formed by reacting with a solution containing 2 wt % galactomannan, 1% hydroxy propyl methyl cellulose and piperlongumine (8.5-10 wt % of that of sorafenib) by stirring for another 10 minutes and then purified by filtration or centrifugation and lyophilized.

### Example 2

Pharmacokinetic Study of Phycocyanin-Sorafenib Nano Formulation-1 with Stirrer Preparation

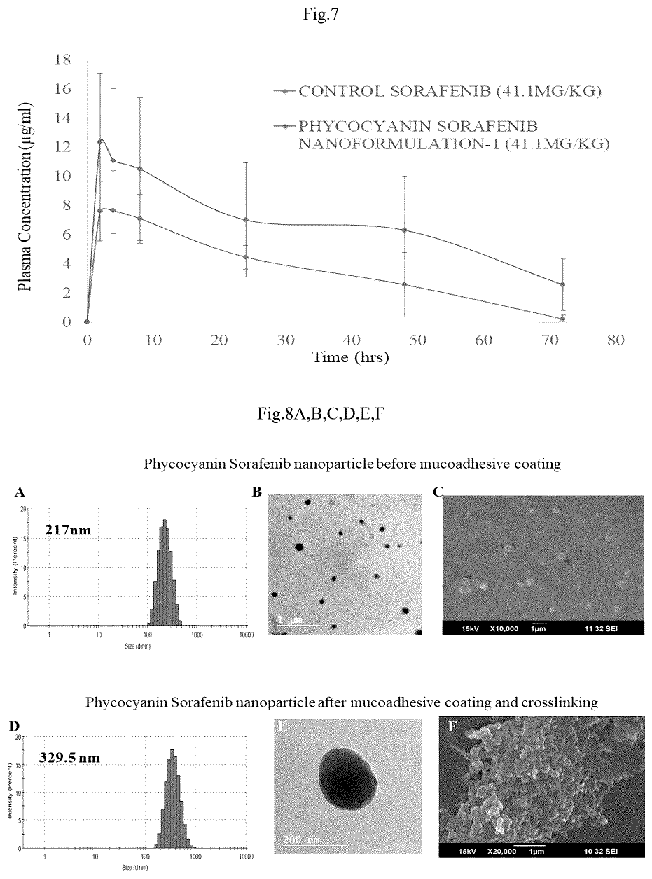

In single dose pharmacokinetic study of phycocyanin-sorafenib nano formulation-1, the plasma concentration of sorafenib for phycocyanin-sorafenib nano formulation-1 was found to be double that of the control sorafenib at same dose of 41.1 mg/kg (FIG. 7).

### Example 3

Preparation and Characterisation of Phycocyanin-Sorafenib Nano formulation-2 Using High-Pressure Homogenization Method

Phycocyanin (PC) is dissolved in water at a concentration of 5 mg/ml. Polymers; PEG-400 (10 wt % of the protein) and PCL-PVA-PEG block copolymer (50% by weight of protein) is blended to the protein solution while stirring for total 10 minutes. Sorafenib tosylate (1:1 ratio with respect to PC) is dissolved in DMSO. This mixture is added to PC solution drop-wise and stirred continuously for precipitation of nanoparticles at room temperature. The prepared suspension is transferred to high pressure homogeniser (HPH) and homogenised under 20,000psi for 20 cycles. This homogenization process helps in the formation of nanoparticles in the size range of 200-220 nm (FIGS. 8A, B & C). In the next step, the nanoparticles are coated with the blend of piperine (8.5-10 wt % with respect to sorafenib), 2% sodium alginate and 1% hydroxy propyl methyl cellulose and homogenized at 20,000 psi for 10 minutes (FIGS. 8 D, E & F). This mixture is preferably cross-linked using 5% calcium chloride solution. The precipitate is continuously stirred for 15 mins, purified by filtration or centrifugation and lyophilized. This final formulation is referred as phycocyanin-sorafenib nano formulation-2. The size, zeta potential, encapsulation efficiency and loading efficiency of different batches of Phycocyanin sorafenib nano formulation-2 is shown in FIG. 15.

### Example 4

### Pharmacokinetic Study of Phycocyanin-Sorafenib Nano Formulation-2

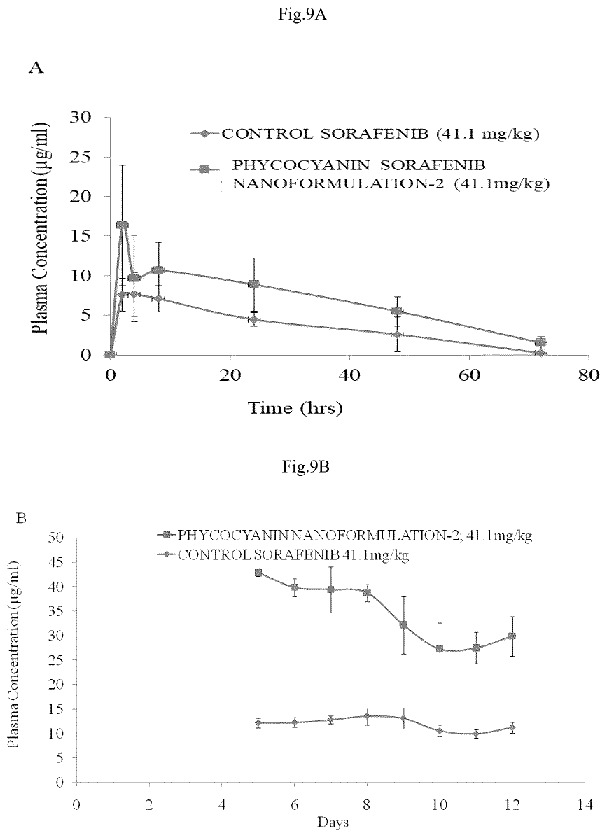

The single dose pharmacokinetic study of final Phycocyanin-sorafenib nano formulation-2 administered orally at a dose of 41.1 mg/kg shows 2-fold enhancement in plasma concentration of sorafenib in (FIG. 9A). For multi dose study animals were orally administered with control sorafenib and phycocyanin sorafenib nano formulation-2 for a period of 12 days. After initial dosing for 4 days, blood draw was started from 5th day till 12th day via tail vein. Plasma concentration of sorafenib was found to be enhanced by 2-3 fold for phycocyanin-sorafenib nano formulation-2 compared to the control sorafenib (FIG. 9B).

### Example 5

### Anti Tumor Study of Phycocyanin Sorafenib Nano Formulation-2

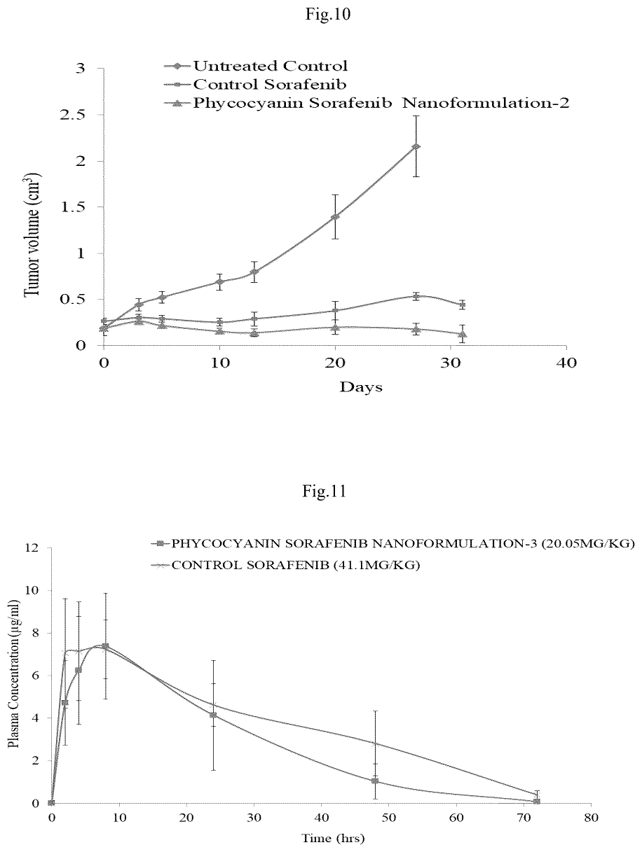

Considering the potency of Sorafenib towards FLT-3-ITD positive Acute Myeloid Leukemia (AML), a preliminary evaluation of the phycocyanin-sorafenib nano formulation towards AML xenograft using MV-411 cells in Nude mice (Swiss Albino nu/nu) models has been attempted. FIG. 10 showed the tumor reduction profile of MV-411 subcutaneous AML xenograft upon treatment with phycocyanin-sorafenib nano formulation with respect to control sorafenib. It was observed that alternate day dosing of 41.1 mg/kg of phycocyanin-sorafenib nano formulation-2 effectively improved antitumor efficacy than the control sorafenib resulting in complete elimination of the subcutaneous tumors post 15 day treatment.

### Example 6

### Preparation of Phycocyanin Sorafenib Nano Formulation-3 Using Stirrer Method

Phycocyanin is dissolved in water at a concentration of 5 mg/ml. Polymers; PEG-400 (10 wt % of the protein) and PCL-PVA-PEG block copolymer (50% by weight of protein) is blended to the protein solution while stirring for total 10 minutes. Sorafenib tosylate (1:1 ratio with respect to protein) is dissolved in DMSO. This mixture is added to composite carrier solution drop-wise and stirred continuously for precipitation of nanoparticles at room temperature at 1200 rpm for 10 mimutes.

In the next step, the nanoparticles are coated with a layer formed by reacting with a solution containing 2 wt % galactomannan, 1% hydroxy propyl methyl cellulose by stirring for another 10 minutes and then purified by filtration or centrifugation and lyophilized.

### Example 7

### Pharmacokinetic Study of Phycocyanin Sorafenib Nano Formulation-3

In single dose pharmacokinetic study of phycocyanin-sorafenib nano formulation-3, the plasma concentration of sorafenib for phycocyanin-sorafenib nano formulation-3 was found to be equivalent to the control sorafenib at half the dose (FIG. 11). The dose for control sorafenib 41.1 mg/kg and the dose for phycocyanin-sorafenib nano formulation-3 was 20.05 mg/kg.

### Example 8

### Preparation of Albumin Sorafenib Nano Formulation-1 Using High Pressure Homogenization Method

Bovine serum albumin (BSA) is dissolved preferably in water at a concentration of 5 mg/ml. Polymers; PEG-400 (10 wt % of the protein) and PCL-PVA-PEG block copolymer (50% by weight of protein) is blended to the protein solution while stirring for total 10 minutes. Sorafenib tosylate (1:1 ratio with respect to BSA) is dissolved in DMSO. This mixture is added to BSA solution drop-wise and stirred continuously for precipitation of nanoparticles at room temperature. The prepared suspension is transferred to high pressure homogeniser (HPH) and homogenised under 20,000 psi for 20 cycles which help to form nanoparticles. In the next step, the nanoparticles are coated with the blend of piperlongumine (8.5-10% w.r.t drug), 2% galactomannan and 1% hydroxy propyl methyl cellulose solution and stirred for 10 minutes. The precipitate is purified by filtration or centrifugation and lyophilized. This final formulation is referred as Albumin sorafenib nano formulation-1.

### Example 9

### Pharmacokinetic Study of Albumin Sorafenib Nano Formulation-1 in Healthy SD Rats

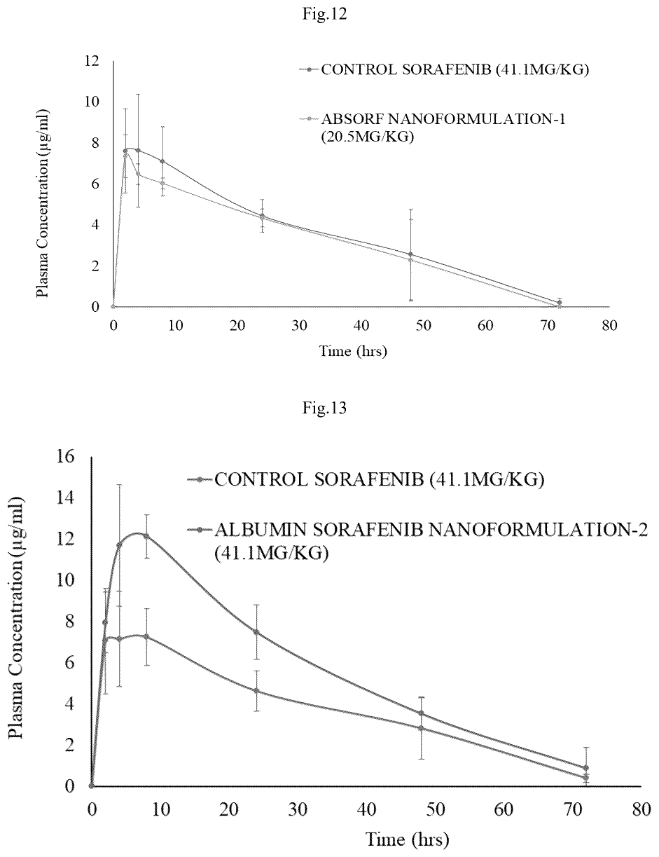

In single dose pharmacokinetic study of Albumin-sorafenib nano formulation-1 (ABSORF NANOFORMULATION-1), the plasma concentration of sorafenib for albumin sorafenib nano formulation-1 was found to be equivalent to the control sorafenib at half the dose (FIG. 12). The dose for control sorafenib 41.1 mg/kg and the dose for albumin sorafenib nano formulation-1 was 20.05 mg/kg.

### Example 10

### Preparation of Albumin Sorafenib Nano Formulation-2 Using Stirrer Method

Bovine serum albumin (BSA) is dissolved preferably in MilliQ water at a concentration of 5 mg/ml. Polymers; PEG-400 (10 wt % of the protein) and PCL-PVA-PEG block copolymer (50% by weight of protein) is blended to the protein solution while stirring for total 10 minutes. Sorafenib tosylate (1:1 ratio with respect to BSA) is dissolved in DMSO. This mixture is added to BSA solution drop-wise and stirred continuously for precipitation of nanoparticles at room temperature at 1200 rpm for 10 mimutes. In the next step, the nanoparticles are coated with blend of piperine (8.5-10% with respect to sorafenib), 2 wt % sodium alginate and 1% hydroxy propyl methyl cellulose solution and stirred for another 10 minutes. This mixture is further cross-linked using 5% calcium chloride solution and stirred continuously for 15 mins and then purified by filtration or centrifugation and lyophilized. This final formulation is referred as Albumin sorafenib nano formulation-2.

### Example 11

Pharmacokinetic Study of Albumin Sorafenib Nano Formulation-2 Using Stirrer Method

In single dose pharmacokinetic study of Albumin-sorafenib nano formulation-2, the plasma concentration of sorafenib for albumin sorafenib nano formulation-2 was found to be double that of control sorafenib at same dose (FIG. 13). The dose for control sorafenib and albumin sorafenib nano formulation-2 was 41.1 mg/kg.

### Example 12

### Preparation of Albumin Sorafenib Nano Formulation-2 Using High Pressure Homogenization

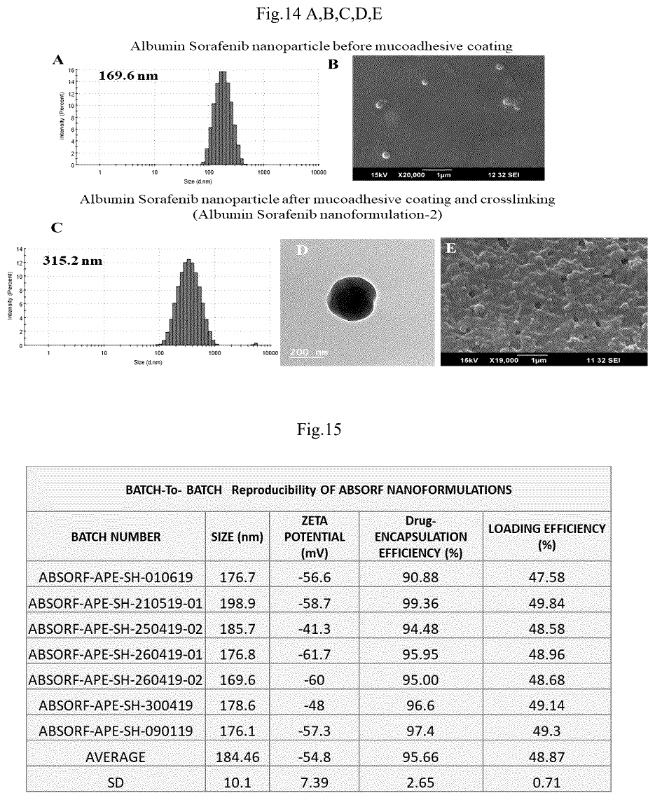

Bovine serum albumin (BSA) is dissolved preferably in water at a concentration of 5 mg/ml. Polymers; PEG-400 (10 wt % of the protein) and PCL-PVA-PEG block copolymer (50% by weight of protein) is blended to the protein solution while stirring for total 10 minutes. Sorafenib tosylate (1:1 ratio with respect to BSA) is dissolved in DMSO. This mixture is added to BSA solution drop-wise and stirred continuously for precipitation of nanoparticles at room temperature. The prepared suspension is transferred to high pressure homogeniser (HPH) and homogenised at 20,000 psi for 20 cycles. This homogenization process helps in obtaining nanoparticles in the size range of 160-170 nm (FIGS. 14A & B). In the next step, the nanoparticles are coated with the blend of piperine (8.5-10% with respect to sorafenib), 2 wt % sodium alginate and 1% hydroxypropyl methyl cellulose and homogenized at 20,000 psi for 10 minutes (FIG. 14C & D). This mixture is further cross-linked using 5% calcium chloride solution. The precipitate is continuously stirred for 15 mins, purified by filtration or centrifugation and lyophilized. The size, zeta potential, encapsulation efficiency and loading efficiency of different batches of Albumin sorafenib nano formulation-2 is shown in FIG. 15.

### Example 13

Detachment Force Study of Different Albumin Sorafenib (ABSORF) Nano Formulation

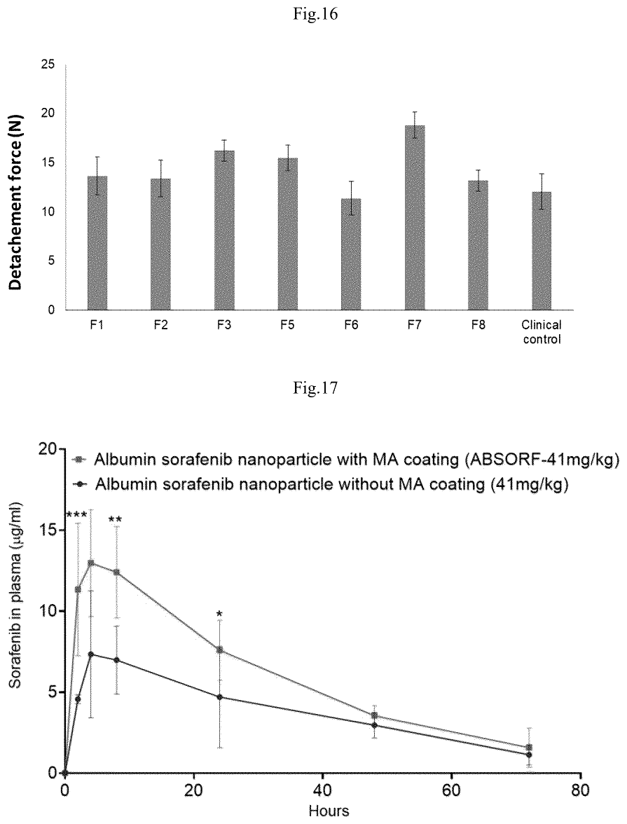

Mucoadhesive polymers in a range of concentrations were coated and crosslinked around ABSORF nano formulation to micro matrix structure to get formulation F1 to F8. The prepared material in solution was placed between two intestinal samples, pressed and force required to detach them is found out. The higher detachment force implies good mucoadhesive property. FIG. 16 shows the detachment force required by ABSORF nano formulation coated with mucoadhesive layers with a range of concentration. Mucoadhesive polymers used in F1 to F8 comprises alginate (low and medium viscosity), HPMC and CMC, in varying combinations where the total amount of mucoadhesive polymers is 100 mg. Clinical control used here is sorafenib tablet which is currently given to patients. F7 shows a better muco-adhesion compared to all the others, which is Albumin sorafenib nano formulation-2.

### Example 14

### Pharmacokinetic Study of Albumin Sorafenib Nano Formulation-2

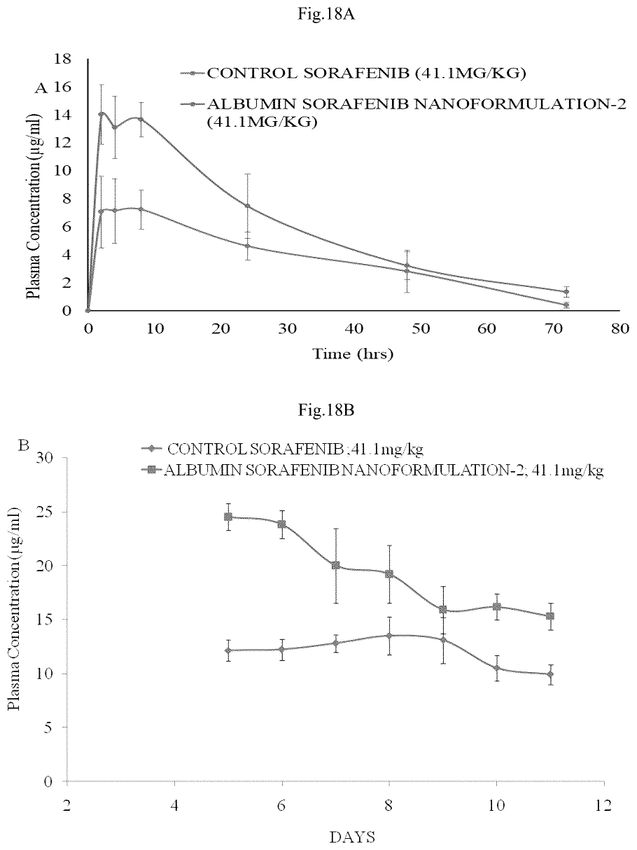

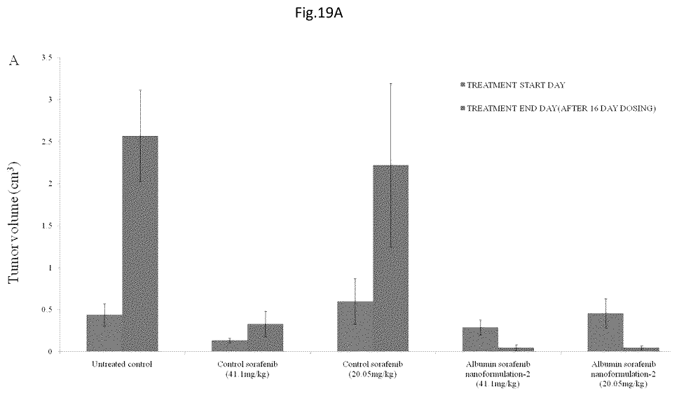

The effect of mucoadhesive and muco-penetrating polymer coating is shown by single dose pharmacokinetic study of Albumin sorafenib nanoparticle with and without mucoadhesive and muco-penetrating polymer coatings in healthy SD rats (FIG. 17). The nano formulation with and without mucoadhesive polymeric layer is compared with control sorafenib at a dose of 41.1 mg/kg (human equivalent dose 400 mg) administered orally. The single dose pharmacokinetic study of final Albumin-sorafenib nano formulation-2 administered orally at a dose of 41.1 mg/kg shows 1.5-2 fold enhancement in plasma concentration of sorafenib (FIG. 18A).

For multi dose study animals were orally administered with control sorafenib and albumin sorafenib nano formulation-2 for a period of 12 days. After initial dosing for 4 days, blood draw was started from 5th day till 12th day via tail vein. Plasma concentration of sorafenib was found to be enhanced by 2 fold for albumin nano formulation-2 compared to the control sorafenib (FIG. 18B)

### Example 15

### Antitumor Study of Albumin Sorafenib Nano Formulation-2

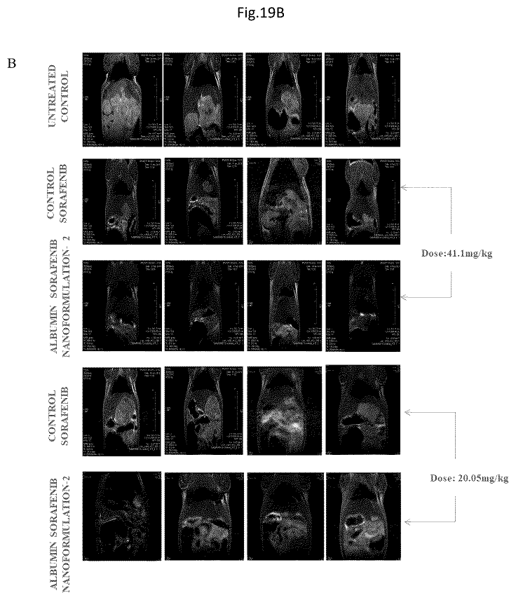

Anti tumor efficacy of albumin sorafenib nano formulation 2 is tested in an orthotopic liver tumor model in Sprague Dawley rats. To develop the orthotopic tumor, rat hepatoma cells N1S1 (107 cells/100 ml of basal media) is surgically injected into the left lobe of liver. Tumor growth is monitored using MRI and after 7 days of induction the animals are orally treated with control sorafenib and albumin sorafenib nano formulation (41.1 mg/kg & 20.05 mg/kg) for 16 days (FIG. 19A). The representative MRI images are shown in FIG. 19B.

### Example 16

### Preparation of Albumin Sorafenib Nano Formulation-2 Using Colloidal Wet Milling Method

Bovine serum albumin (BSA) is dissolved preferably in water at a concentration of 5 mg/ml. Polymers; PEG-400 (10 wt % of the protein) and PCL-PVA-PEG block copolymer (50% by weight of protein) is blended to the protein solution while stirring for total 10 minutes. Sorafenib tosylate (1:1 ratio with respect to BSA) is dissolved in DMSO. This mixture is added to BSA solution drop-wise and stirred continuously for precipitation of nanoparticles at room temperature. The prepared suspension is transferred to colloidal milling/dispersion unit for 20 cycles. In the next step, the nanoparticles are coated with the blend of piperine (8.5-10% with respect to sorafenib), 2 wt % sodium alginate and 1% hydroxy propyl methyl cellulose and mixed for 10 minutes. This mixture is further cross-linked using 5% calcium chloride solution. The precipitate is continuously stirred for 15 mins, purified by filtration or centrifugation and lyophilized.

### Example 17

### Preparation of Albumin Sorafenib Nano Formulation-3 Using High Pressure Homogenization Method

Bovine serum albumin (BSA) is dissolved preferably in water at a concentration of 5 mg/ml Polymers; PEG-400 (10 wt % of the protein) and PCL-PVA-PEG block copolymer (50% by weight of protein) is blended to the protein solution while stirring for total 10 minutes. Sorafenib tosylate (1:1 ratio with respect to BSA) is dissolved in DMSO. This mixture is added to BSA solution drop-wise and stirred continuously for precipitation of nanoparticles at room temperature. The prepared suspension is transferred to high pressure homogeniser (HPH) and homogenised under 20,000 psi for 20 cycles which help to form nanoparticles. In the next step, the nanoparticles are coated with the blend of 3-acetyl-11-keto-β-boswellic acid (8.5-10% with respect to sorafenib), 2% galactomannan and 1% hydroxy propyl methyl cellulose solution and stirred for 10 minutes. The precipitate is purified by filtration or centrifugation and lyophilized. This formulation is referred as Albumin sorafenib nano formulation-3 (ABSORF NANOFORMULATION-3).

### Example 18

### Pharmacokinetic Study Albumin Sorafenib Nano Formulation-3 in Healthy SD Rats

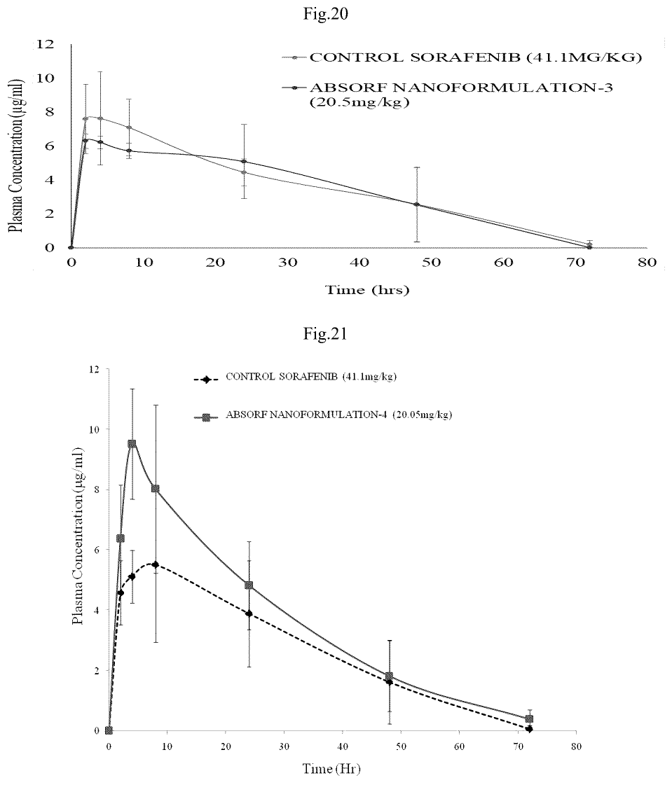

In single dose pharmacokinetic study of Albumin-sorafenib nano formulation-3, the plasma concentration of sorafenib for albumin sorafenib nano formulation-3 was found to be equivalent to the control sorafenib at half the dose (FIG. 20). The dose for control sorafenib 41.1 mg/kg and the dose for albumin sorafenib nano formulation-3 was 20.05 mg/kg.

### Example 19

Preparation of Albumin Sorafenib Nano Formulation-4 using Stirrer Method

Bovine serum albumin (BSA) is dissolved preferably in MilliQ water at a concentration of 5 mg/ml. Polymers; PEG-400 (10 wt % of the protein) and PCL-PVA-PEG block copolymer (50% by weight of protein) is blended to the protein solution while stirring for total 10 minutes. Sorafenib tosylate (1:1 ratio with respect to BSA) is dissolved in DMSO. This mixture is added to BSA solution drop-wise and stirred continuously for precipitation of nanoparticles at room temperature at 1200 rpm for 10 mimutes. In the next step, the nanoparticles are coated with blend of 2 wt % sodium alginate and 1% hydroxy propyl methyl cellulose solution and stirred for another 10 minutes. This mixture is further cross-linked using 5% calcium chloride solution and stirred continuously for 15 mins and then purified by filtration or centrifugation and lyophilized. This final formulation is referred as Albumin sorafenib nano formulation-4 (ABSORF NANOFORMULATION-4).

### Example 20

Pharmacokinetic Study of Albumin Sorafenib Nano Formulation-4 in Healthy SD Rats In single dose pharmacokinetic study of Albumin-sorafenib nano formulation-4, the plasma concentration of sorafenib for albumin sorafenib nano formulation-4 was found to be equivalent to the control sorafenib at half the dose (FIG. 21). The dose for control sorafenib 41.1 mg/kg and the dose for albumin sorafenib nano formulation-4 was 20.05 mg/kg.

### Example 21

Preparation of Albumin sorafenib nano formulation-5 using stirrer method Bovine serum albumin (BSA) is dissolved preferably in water at a concentration of 5 mg/ml. Polymers; PEG-400 (10 wt % of the protein) and PCL-PVA-PEG block copolymer (50% by weight of protein) is blended to the protein solution while stirring for total 10 minutes. Sorafenib tosylate (1:1 ratio with respect to protein) is dissolved in DMSO. This mixture is added to composite carrier solution drop-wise and stirred continuously for precipitation of nanoparticles at room temperature at 1200 rpm for 10 mimutes. In the next step, the nanoparticles are coated with a layer formed by reacting with a solution containing 2 wt % galactomannan, 1% hydroxy propyl methyl cellulose by stirring for another 10 minutes and then purified by filtration or centrifugation and lyophilized.

### Example 22

Preparation of Albumin-Temozolomide Nano Formulation Using Stirrer Method

Bovine serum albumin (BSA) is dissolved preferably in water at a concentration of 5 mg/ml. Polymers; PEG-400 (10 wt % of the protein) and PCL-PVA-PEG block copolymer (50% by weight of protein) is blended to the protein solution while stirring for total 10 minutes. Temozolomide (1:1 ratio with respect to BSA) is dissolved in DMSO. This mixture is added to composite carrier solution drop-wise and stirred continuously for precipitation of nanoparticles at room temperature at 1200 rpm for 10 mimutes. In the next step, the nanoparticles are coated with the blend of piperine (8.5-10% with respect to temozolomide), 2 wt % galactomannan and 1% hydroxy propyl methyl cellulose by stirring for another 10 minutes and then purified by filtration or centrifugation and lyophilized.

### Example 23

### Pharmacokinetic Study of Albumin-Temozolomide Nano Formulation in Healthy SD Rats

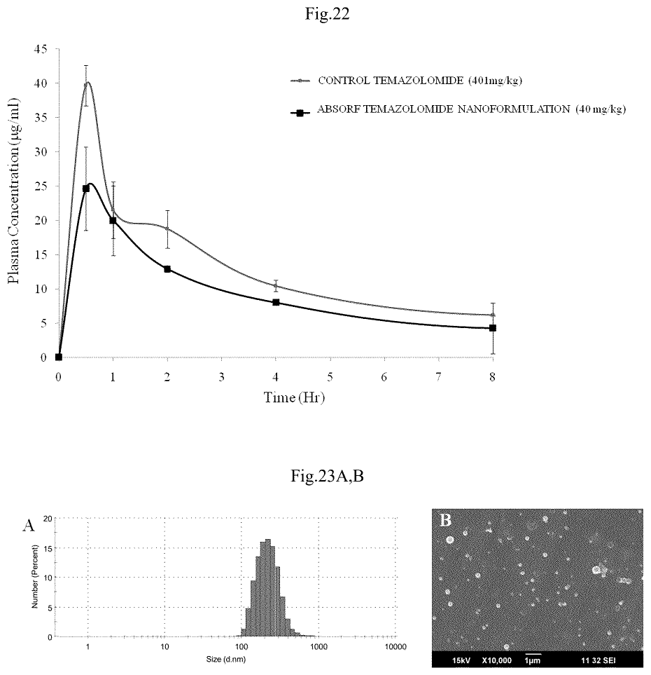

In single dose pharmacokinetic study of Albumin-temozolomide nano formulation, the plasma concentration of temozolomide for albumin-temozolomide nano formulation was found to be ˜2 fold to the control temozolomide at same dose (FIG. 22). The dose for control temozolomide 40 mg/kg and the dose for albumin-temozolomide nano formulation was 40 mg/kg.

### Example 24

Preparation of Casein-lenvatinib nano formulation using stirrer method Sodium caseinate is dissolved preferably in water at a concentration of 5 mg/ml. Polymers; PEG-400 (10 wt % of the protein) and PCL-PVA-PEG block copolymer (50% by weight of protein) is blended to the protein solution while stirring for total 10 minutes. Lenvatinib is dissolved in DMSO. This mixture is added to composite carrier solution drop-wise and stirred continuously for precipitation of nanoparticles at room temperature at 1200 rpm for 10 mimutes. In the next step, the nanoparticles are coated with the blend of piperine (8.5-10% with respect to lenvatinib), 2 wt % sodium alginate solution and 1% hydroxy propyl methyl cellulose by stirring for another 10 minutes and then purified by filtration or centrifugation and lyophilized. Size distribution shows particle in the range of 294.6 nm with spherical morphology (FIG. 23).

### Example 25

### Preparation of Zein-Regorafenib Nano Formulation Using Stirrer Method

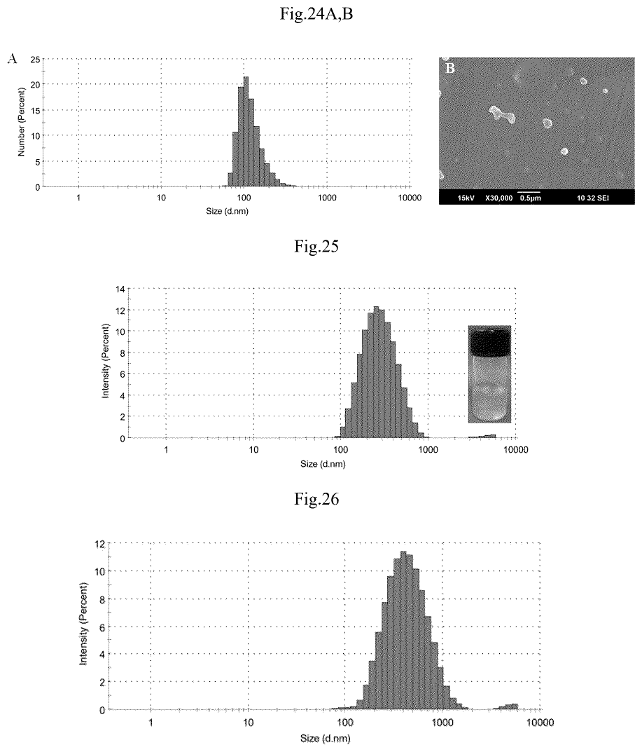

Zein is dissolved preferably in DMSO at a concentration of 5 mg/ml. Polymer; PCL-PVA-PEG block copolymer (50% by weight of protein) is blended to the protein solution while stirring for total 10 minutes. Regorafinib is also dissolved in DMSO. This mixture is stirred for 15 minutes and added drop-wise to water containing PEG-400 (10wt % of the zein) and stirred continuously for precipitation of nanoparticles (RT, 1200 rpm for 10 mimutes). In the next step, the nanoparticles are coated with the blend of piperine (8.5-10% with respect to regorafenib), 2 wt % sodium alginate solution and 1% hydroxy propyl methyl cellulose by stirring for another 10 minutes and then purified by filtration or centrifugation and lyophilized. Size distribution shows particle in the range of 166.4 nm with spherical morphology (FIG. 24).

### Example 26

### Preparation of Albumin-Cabozantinib Nano Formulation Using Stirrer Method

Bovine serum albumin (BSA) is dissolved preferably in water at a concentration of mg/ml. Polymers; PEG-400 (10 wt % of the protein) and PCL-PVA-PEG block copolymer (50% by weight of protein) is blended to the protein solution while stirring for total 10 minutes. Cabozantinib is dissolved in DMSO. This mixture is added to composite carrier solution drop-wise and stirred continuously for precipitation of nanoparticles at room temperature at 1200 rpm for 10 mimutes. In the next step, the nanoparticles are coated with the blend of piperine (8.5-10% with respect to cabozantinib), 2 wt % sodium alginate solution and 1% hydroxy propyl methyl cellulose by stirring for another 10 minutes and then purified by filtration or centrifugation and lyophilized. Size distribution shows particle in the range of 256.4 nm (FIG. 25).

### Example 27

### Preparation of Albumin-Lopinavir Nano Formulation Using Stirrer Method

Bovine serum albumin (BSA) is dissolved preferably in water at a concentration of 5 mg/ml. Polymers; PEG-400 (10 wt % of the protein) and PCL-PVA-PEG block copolymer (50% by weight of protein) are blended to the protein solution while stirring for total 10 minutes. Lopinavir is dissolved in DMSO and PEG-400. This mixture is added to composite carrier solution drop-wise and stirred continuously for precipitation of nanoparticles at room temperature at 1200 rpm for 10 mimutes. In the next step, the nanoparticles are coated with the blend of piperine (8.5-10% with respect to lopinavir), 2 wt % sodium alginate solution and 1% hydroxy propyl methyl cellulose by stirring for another 10 minutes and then purified by filtration or centrifugation and lyophilized. Size of prepared nano formulation is in the range of 387.8 nm (FIG. 26).

### Example 28

### Preparation of Albumin-Amphotericin Nano Formulation Using Stirrer Method

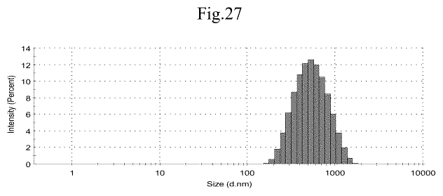

Bovine Serum Albumin (BSA) is dissolved Preferably in Water at a Concentration of 5 mg/ml. Polymers; PEG-400 (10 wt % of the protein) and PCL-PVA-PEG block copolymer (50% by weight of protein) is blended to the protein solution while stirring for total 10 minutes. Amphotericin is dissolved in DMSO and PEG-400. This mixture is added to composite carrier solution dropwise and stirred continuously for precipitation of nanoparticles at room temperature at 1200 rpm for 10 mimutes. In the next step, the nanoparticles are coated with the blend of piperine (8.5-10% with respect to amphotericin), 2 wt % sodium alginate solution and 1% hydroxy propyl methyl cellulose by stirring for another 10 minutes and then purified by filtration or centrifugation and lyophilized. Size of prepared nanoformulation is in the range of 437.6 nm (FIG. 27).

1. A formulation comprising:
core nanoparticles and
shell;
wherein said core nanoparticles are coated with one or more layers of said shell; wherein the core nanoparticles comprise drug dissolved in solvent I, carrier protein dissolved in solvent II, polymer; wherein the one or more layers of shell are made of substances comprising muco-adhesives, release modifiers.

2. The formulation of claim 1, wherein said substances further comprise bioavailability enhancers and cross-linking agents.

3. The formulation of claim 1, further comprising binders, fillers, disintegrating agent, glidants, lubricants, sorbents, preservative, excipients, stabilizers, solubilizers and flavouring agents suitable for making capsule or tablet or suspension suitable for oral delivery.

4. The formulation of claim 1, wherein the ratio of drug dissolved in solvent I and carrier protein dissolved in solvent II in the core nanoparticles is 1:1.

5. The formulation of claim 1, wherein the core nanoparticles range in size between 1-1000 nm, and wherein particles in the final formulation range in size between 1-1000 μm.

6. The formulation of claim 1, wherein the polymer is in the range of 10% to 50% by weight of carrier protein in the core nanoparticles; wherein the one or more layers of shell comprise muco-adhesives in the range of 20-40% by weight of drug; release modifiers in the range of 1-15% by weight of drug; bioavailability enhancers in the range of 8.5-10% by weight of drug; and cross-linking agents in the range of 5%-20% by weight of drug.

7. The formulation of claim 1, wherein the drug is present in the range of 5 mg/ml-100 mg/ml of the final formulation.

8. The formulation of claim 1, wherein the drug is selected from anti-cancer drugs comprising small-molecule inhibitors and DNA alkylating agents; anti-fungal drugs, anti-viral drugs; wherein the small-molecule inhibitors comprise sorafenib, sorafenib tosylate, everolimus, rapamycin, tandutinib, sunitinib, lestaurtinib, semaxinib, midostaurin, nilotinib, dasatinib, imatinib, temsirolimus, lapatinib, vorinostat, bosutinib, olaparib, erlotinib, epirubicin, gefitinib, daunorubicin, temozolomide, nintedanib, crizotinib, dabrafenib, vemurafenib, ibrutinib, axitinib, regorafenib, ponatinib, cabozantinib, alectinib, brigatinib, lorlatinib, encorafenib, acalabrutinib, vandetanib, cobimetinib, lenvatinib, binimetinib, ceritinib, pazopanib, tacrolimus; wherein the DNA alkylating agents comprise temozolomide, wherein the anti-fungal drugs comprise amphotericin and anti-viral drugs comprise lopinavir.

9. The formulation of claim 1, wherein the carrier protein comprises human serum albumin, bovine serum albumin, phycocyanin, fibrinogen, collagen, gelatin, casein, mucin, protamine, transferrin, soy protein, apoferritin, ferritin, lectin, lactoferrin, gluten, whey protein, prolamins such as gliadin, hordein, secalin, zein, avenin, or their salts.

10. The formulation of claim 1, wherein the solvent I comprises water or organic solvent comprising dimethyl sulfoxide (DMSO), methanol, ethanol, dimethyl formamide, acetonitrile, dioxane and wherein the solvent II is an organic solvent comprising dimethyl sulfoxide (DMSO), methanol, ethanol, dimethyl formamide, acetonitrile, dioxane or combination of organic solvent and polymer comprising PEG-400.

11. The formulation of claim 1, wherein the polymer is selected from polyethylene glycol 400 (PEG-400), block co-polymer of polyvinyl caprolactum-polyvinyl alcohol-polyethylene glycol (PCL-PVA-PEG) or combination thereof.

12. (canceled)

13. The formulation of claim 1, wherein the muco-adhesives comprises galactomannan, alginate preferably sodium alginate.

14. The formulation of claim 1, wherein the release modifier is selected from methylcellulose (MC), ethylcellulose (EC), hydroxyethyl cellulose (HEC), hydroxypropyl cellulose (HPC), hydroxypropyl methylcellulose (HPMC), hydroxypropyl methacrylate, and carboxymethyl cellulose (CMC), hydroxypropyl methyl cellulose phthalate (HPMCP), polypropylene glycol, poly methyl acrylates selected from Eudragit RS100, RS PO, RS 30D, RL100, RL PO, RL 30D, NE30D, and NE40D.

15. (canceled)

16. The formulation of claim 1, wherein the bioavailability enhancer (106, 306, 506) is selected the group consisting of piperlongumine, piperine, boswellic acid extracts, 3-acetyl-11-keto-β-boswellic acid, and soluplus.

17. (canceled)

18. The formulation of claim 1, wherein the cross-linking agent comprises calcium chloride, or glutaraldehyde.

19. A formulation, as claimed in claim 2, wherein the core nano particle comprises phycocyanin or albumin dissolved in water, polyethylene glycol, PCL-PVA-PEG block copolymer, sorafenib tosylate dissolved in DMSO; wherein phycocyanin or albumin dissolved in water and sorafenib tosylate dissolved in DMSO are present in the ratio of 1:1; and wherein the one or more layers of shell is formed of galactomannan, HPMC, and piperlongumine or sodium alginate, HPMC, piperine, and calcium chloride.

20. (canceled)

21. (canceled)

22. (canceled)

23. A formulation, as claimed in claim 2, wherein the core nano particle comprises of albumin dissolved in water, polyethylene glycol, PCL-PVA-PEG block copolymer, sorafenib tosylate dissolved in DMSO; and wherein albumin dissolved in water and sorafenib tosylate dissolved in DMSO are present in the ratio of 1:1; wherein the one or more layers of shell is formed of sodium alginate, HPMC, and calcium chloride, or 3-acetyl-11-keto-β-boswellic acid, galactomannan, and HPMC.

24. (canceled)

25. (canceled)

26. The formulation of claim 1, wherein the formulation consists of sorafenib as the drug range of 50 mg to 800 mg.

27. The formulation of claim 1, wherein the formulation consists of temozolomide as the drug and in a range of 5 mg to 250 mg.

28. The formulation of claim 1, wherein said formulation is in the form of oral powder formulation.

29-36. (canceled)
